(19) United States
(12) Patent Application Publication
(10) Pub. No.: US 2003/0078372 A1
(43) Pub. Date: Apr. 24, 2003

(54) Process for synthesizing peptides by using a PEG polymer support

(75) Inventor: Yung-Fa Chen, Tainan (TW)

Correspondence Address: COHEN, PONTANI, LIEBERMAN & PAVANE, Suite 1210, 551 Fifth Avenue, New York, NY, 10176, US

(73) Assignee: ScinoPharm Singapore

(21) Appl. No.: 10/247,429

(22) Filed: Sep. 19, 2002

Related U.S. Application Data
(60) Provisional application No. 60/323,461, filed on Sep. 19, 2001.

(51) Int. Cl. C07K 1/04

(52) U.S. Cl. 530/333; 530/334

(57) ABSTRACT

The process enables peptides to be synthesized from amino acids with PEG as the polymer support. The PEG support rendered PEG-peptide conjugate soluble in a common organic solvent for solution phase peptide synthesis, thus the process of this invention has the advantage of a solution phase synthesis. The PEG-peptide conjugate can be isolated in a precipitation-filtration-wash sequence by adding a second solvent; thus the process of this invention also has the advantage of a solid phase peptide synthesis. The peptides obtained by this synthesis route are used, in particular, in pharmaceutical applications.

### RELATED APPLICATIONS

This application claims priority from U.S. Provisional Patent Application Serial No. 60/323,461 which was filed on Sep. 19, 2001.

### SUMMARY OF THE INVENTION

1. The present invention relates to a process for synthesizing peptides from amino acids, with PEG as polymer support.

2. PEG polymer has the advantage of good solubility in many of solvents suitable for peptide synthesis. E.g. Dichloromethane, DMF etc. Thus, the peptide synthetic process involving PEG polymer as support is homogeneous in nature.

3. A homogeneous reaction is known to be more predictable for process scale up. Thus it has the advantage in producing large quantity of peptide by using traditional batch type reactors.

4. PEG suitable for the above purpose includes one end capped with alkyl, alkylaryl; non-capped PEG; and hyper-branched PEG. The molecular weight of the PEG suitable for this application can be in the range of 100 to 100,000. Preferably the molecular weight is between 1,000 to 10,000.

5. The amino acids could be attached directly to PEG by forming an ester linkage between the carboxylic group of amino acids and the hydroxyl group of the PEG.

6. The ester linkage formation and the subsequent peptide bond formation can be achieved by coupling reagents that are commonly used for this purpose. E.g. DCC, DIPC, EDCA. Formation of active esters from HOSu, HOBt, F5PhOH and amino acids is also applicable for this purpose.

7. The amino acids used are protected by t-BOC or FMOC as commonly used in solid phase peptide synthesis. TFA and piperidine standard method are used to de-protect nitrogen-protecting group prior to further chain elongation.

8. The peptide chain propagation was done by repetitive N-deprotection and coupling sequence.

9. The PEG-peptide conjugate can be isolated from a reaction mixture by a precipitation-filtration-wash process. Preferable solvents for PEG precipitation/wash are ether type solvents such as diethyl ether, diisopropyl ether and methyl t-butyl ether.

10. If necessary, PEG-peptide conjugate can be further purified by recrystallization.

11. The precipitation-filtration-wash isolation of this PEG-peptide conjugate gives this process the advantage of a solid phase peptide synthesis.

12. The peptide can be cleaved from PEG support by aqueous metal oxide to release peptide from the PEG support or by ammonia, alkylamine to give a peptide with an amide C terminal. Alternatively, trans-esterification in low alcohol gives a peptide with an ester C terminal.

### Synthetic Route

1. Attach a first N-protected AA to PEG by reacting PEG, DCC and N-protected AA in a suitable solvent.

2. Precipitated DCU was filtered off, adding PEG precipitation solvent, isolate PEG-conjugate by filtration, wash the PEG-conjugate by suitable solvent.

3. De-protection of the N-protecting group of PEG conjugate.

4. Adding PEG precipitation solvent, isolate PEG-conjugate by filtration, wash the PEG-conjugate by suitable solvent.

5. Coupling a second N-protected AA to PEG conjugate with DCC in suitable solvent.

6. Repeating step 2-5 with desired AA sequence.

7. Peptide was cleaved from PEG for purification or further manipulation.

### EXAMPLES

Two synthetic peptides obtained according to the process of the invention are given in the following scheme:

### Example 1

### Deslorclin synthesized by PEG supporting process

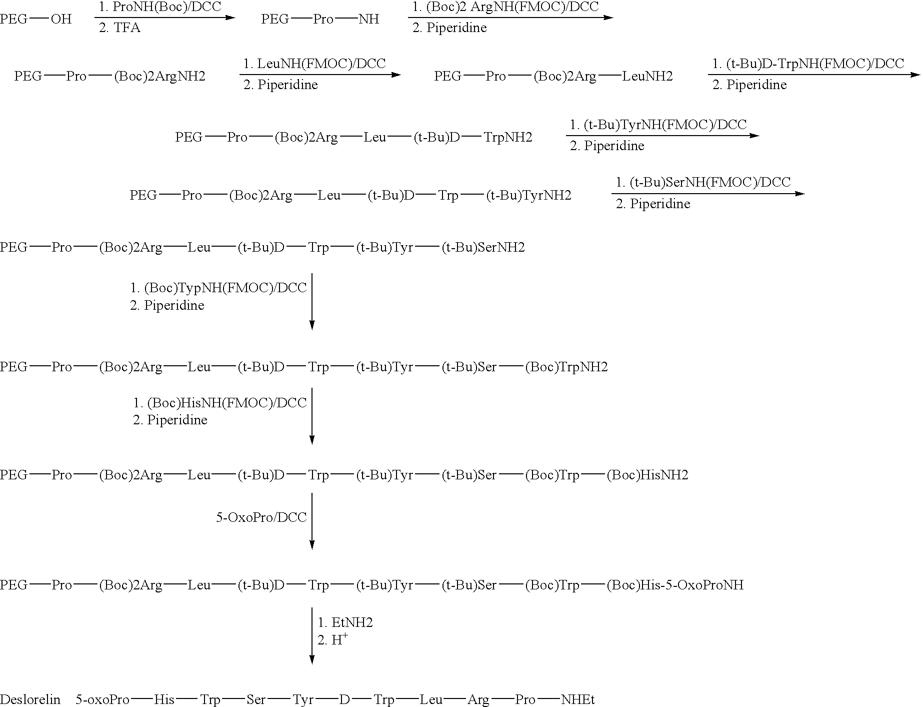

### Example 2

### Eptifibatide synthesized by PEG supporting process

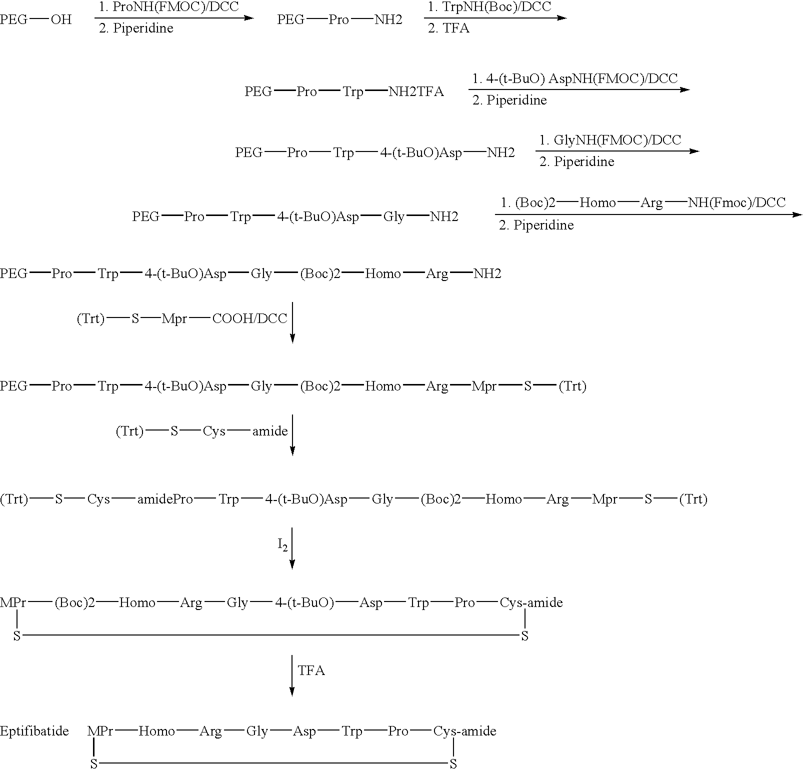

I claim:

1. A method of synthesizing a peptide comprising:
attaching to propylene ethylene glycol at least one amino acid that contains an N-terminal protecting group to produce a propylene ethylene glycol-amino acid conjugate; deprotecting the N-terminal protecting group;
reacting the deprotected propylene ethylene glycol-amino acid conjugate with a coupling reagent to produce a propylene ethylene glycol-peptide conjugate; and cleaving the propylene ethylene glycol-peptide conjugate to release the peptide from the propylene ethylene glycol.
